(19) United States
(12) Patent Application Publication
(10) Pub. No.: US 2015/0033575 A1
(43) Pub. Date: Feb. 5, 2015

(54) DEVICE FOR DRYING AND SANITIZING ITEMS, E.G. SPORTS EQUIPMENT AND CLOTHING

(71) Applicant: Domenico Ciarallo, Kirkland (CA)

(72) Inventor: Domenico Ciarallo, Kirkland (CA)

(21) Appl. No.: 13/956,570

(22) Filed: Aug. 1, 2013

(51) Int. Cl. F26B 25/18; A61L 9/20

(52) U.S. Cl. CPC F26B 25/18; A61L 9/20
USPC 34/275; 34/239

(57) ABSTRACT

A dryer for sporting equipment and clothing includes a free-standing tripod provided with a stand at a lower end thereof, a rack at an upper end of the free-standing tripod, an electric forced air motor located at an upper end of the stand and lower than the rack, and a zippered bag. The outlet of the forced air motor is positioned so that air provided thereby displaces upwards. The zippered bag is placed over the rack and falls up to the forced air motor. The central mast of the free-standing tripod includes a UV light for sanitizing the equipment. Therefore, sporting equipment and clothing can be hung from the rack and within the bag so as to be dried by air provided by the forced air motor.

### FIELD

The present subject-matter relates to the drying of items and, more particularly, to the drying of sporting equipment after it has been used, as it is wet.

### INTRODUCTION

When practising sports, the sporting equipment as well as the user's clothing usually get wet. Properly drying such equipment and clothing is desirable to maintain the same in good condition and also so as to quickly be ready to be used again.

### SUMMARY

It would thus be desirable to be provided with a device for drying and optionally sanitizing sporting equipment and clothing.

The embodiments described herein provide in one aspect a drying device for items such as sporting equipment and clothing, comprising a free-standing structure, a rack at an upper end of the free-standing structure, a forced air motor at a lower end of the free-standing structure, and an enclosure extending outwardly of the rack and downwardly towards the forced air motor, sporting equipment and/or clothing being adapted to be hung from the rack and inwardly of the enclosure, whereby air provided by the forced air motor flows on the sporting equipment and/or clothing thereby at least partly drying the same.

The embodiments described herein provide in another aspect a drying device for items, comprising a support structure, a rack at an upper end of the structure, a blower at a lower end of the structure, and an enclosure extending outwardly of the rack and downwardly towards the blower, the items being adapted to be hung from the rack and inwardly of the enclosure, whereby air provided by the blower acts on the items for at least partly drying the same.

### DRAWINGS

For a better understanding of the embodiments described herein and to show more clearly how they may be carried into effect, reference will now be made, by way of example only, to the accompanying drawings which show at least one exemplary embodiment, and in which:

FIG. 1 is a perspective view of a first exemplary device for drying and sanitizing sport equipment and clothing, which is shown without a cover thereof;

FIG. 2 is a perspective view of the device of FIG. 1, but showing equipment hanging from the device;

FIG. 3 is a perspective view of the device of FIG. 1, but shown with the cover thereof;

FIGS. 4, 5, 6, 7 and 8 are respectively front elevation, right side, rear elevation, top plan and bottom plan views of the device of FIG. 1; and

FIG. 9 is a perspective view of a second exemplary device for drying sport equipment and clothing, which is shown without a cover thereof.

### DESCRIPTION OF VARIOUS EMBODIMENTS

It will be appreciated that, for simplicity and clarity of illustration, where considered appropriate, reference numerals may be repeated among the figures to indicate corresponding or analogous elements or steps. In addition, numerous specific details are set forth in order to provide a thorough understanding of the exemplary embodiments described herein. However, it will be understood by those of ordinary skill in the art that the embodiments described herein may be practiced without these specific details. In other instances, well-known methods, procedures and components have not been described in detail so as not to obscure the embodiments described herein. Furthermore, this description is not to be considered as limiting the scope of the embodiments described herein in any way but rather as merely describing the implementation of the various embodiments described herein.

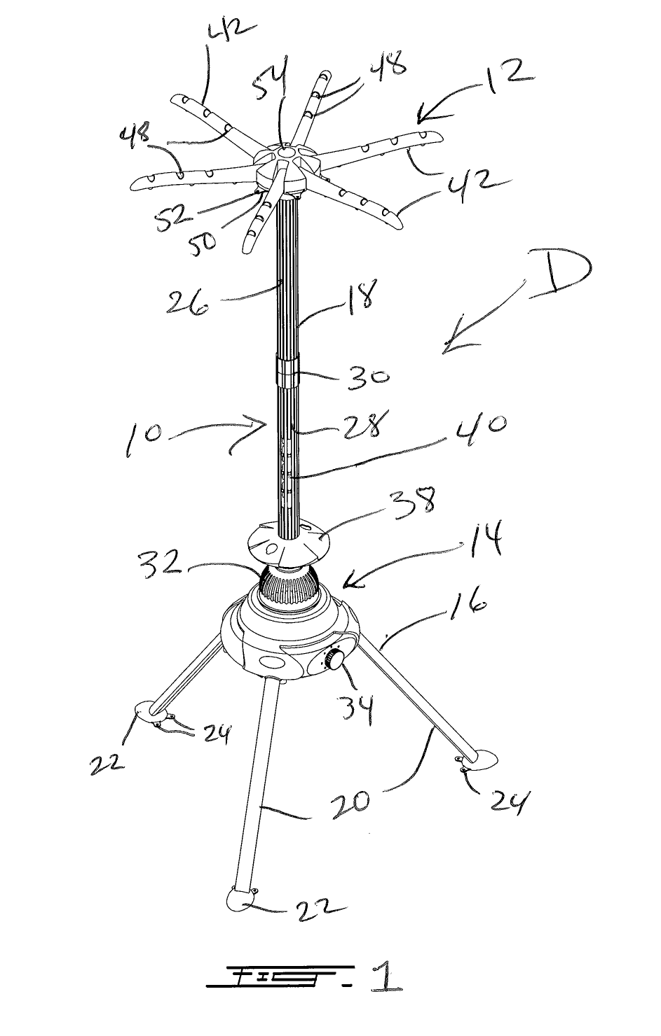

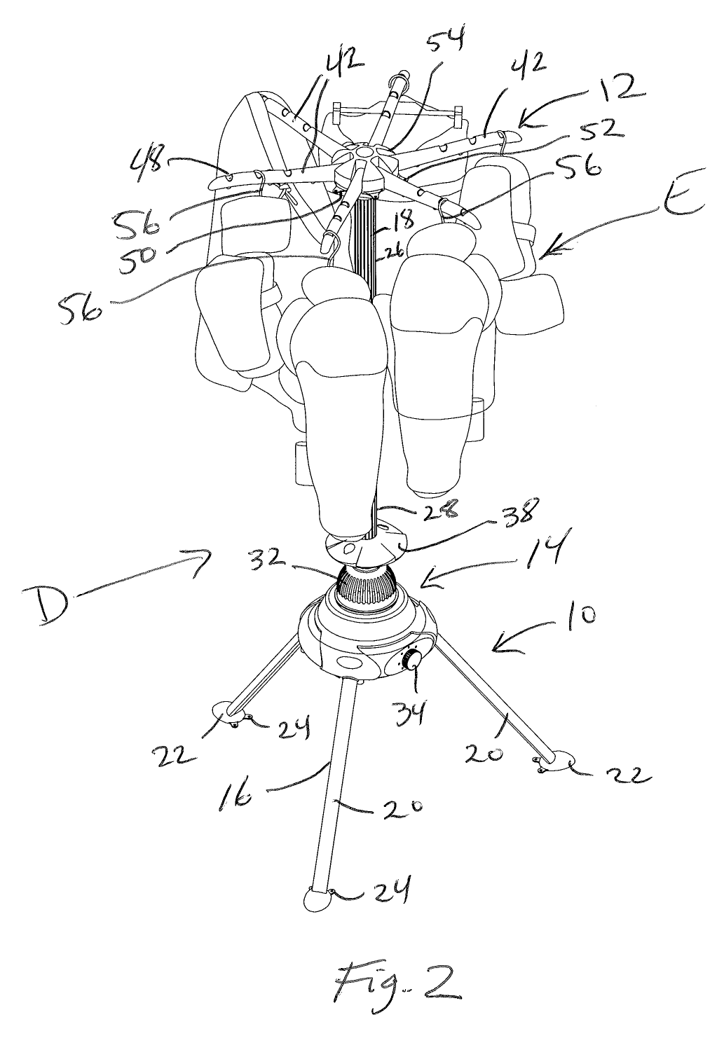

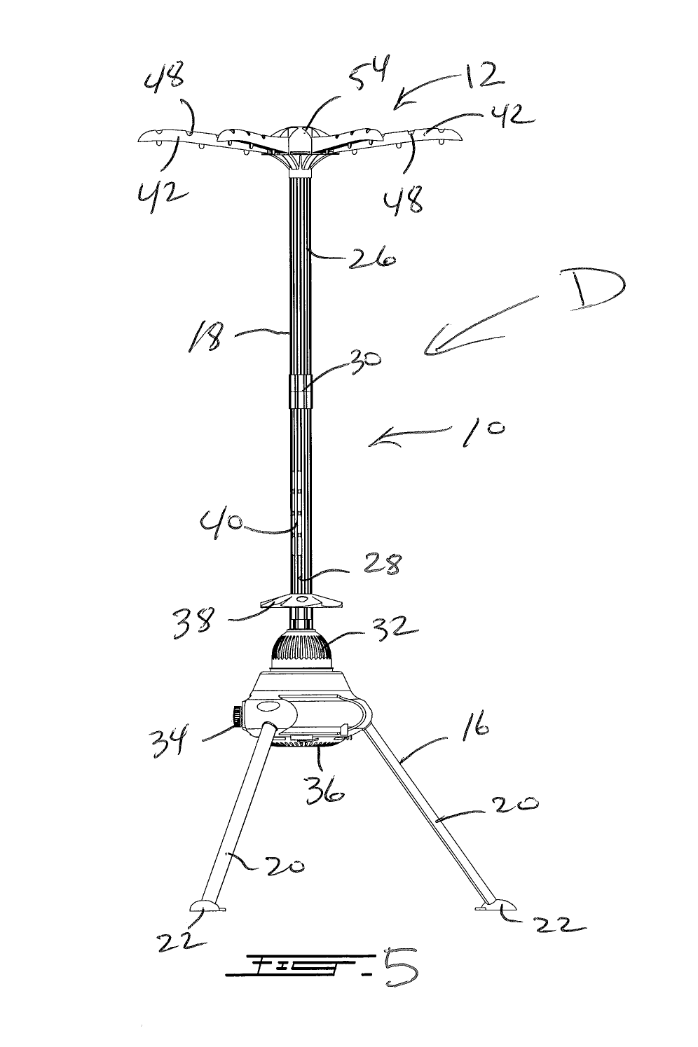

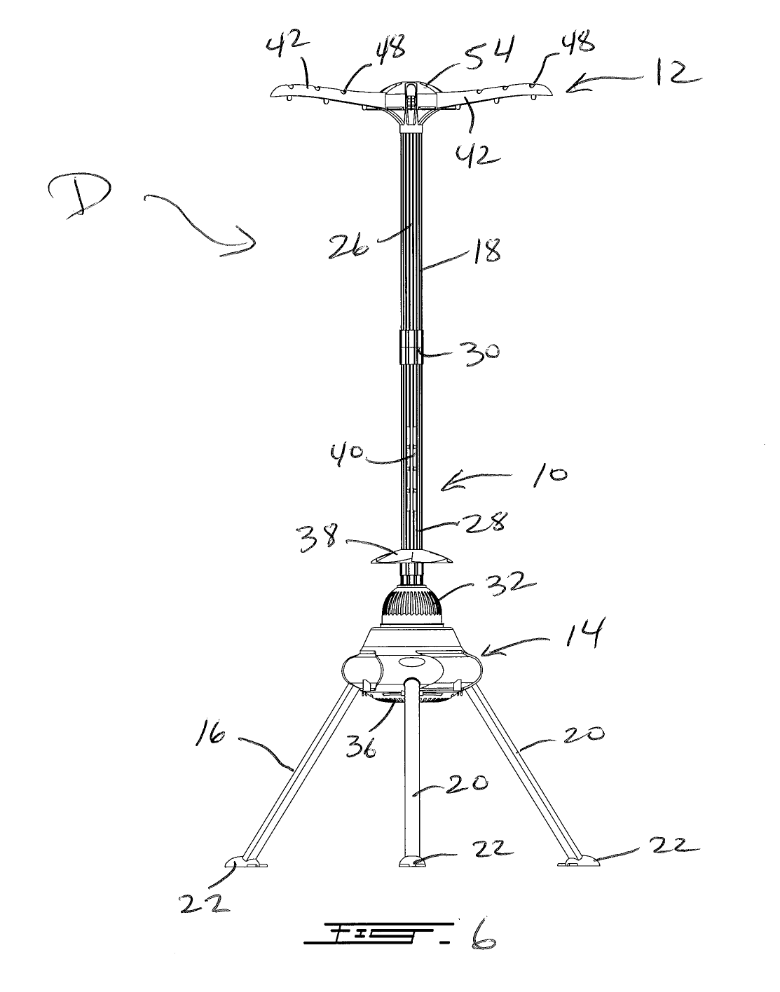

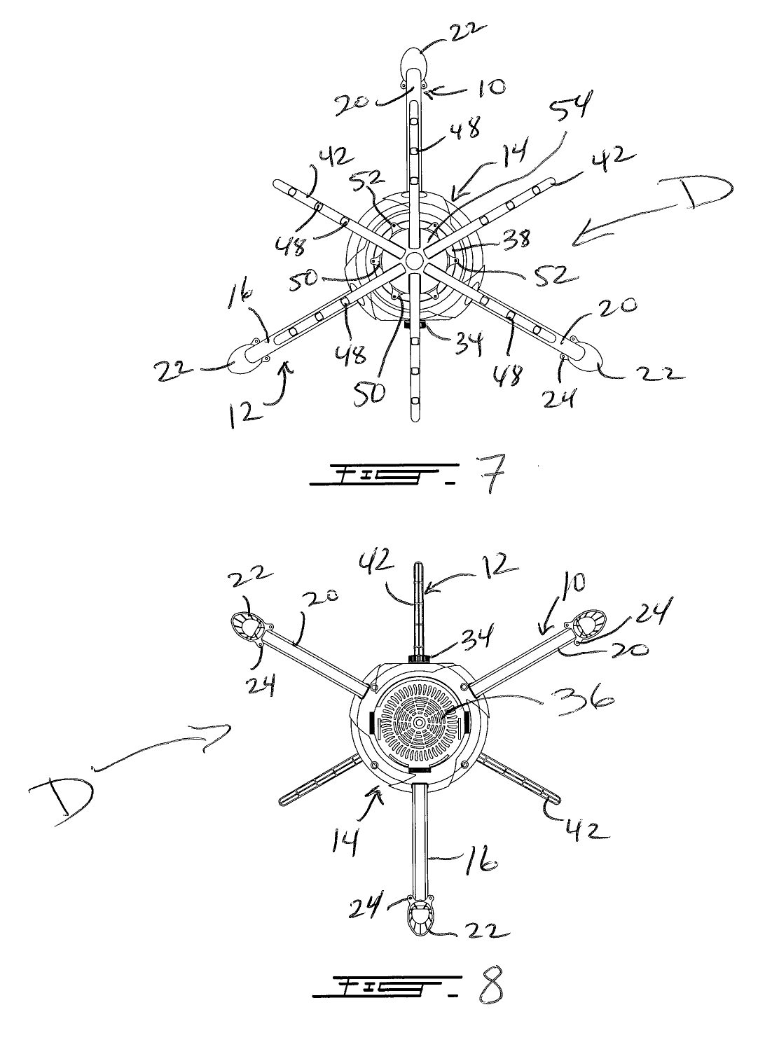

Referring now to FIGS. 1 to 8, therein illustrated is a device D for drying and sanitizing sport equipment and clothing. Equipment and clothing are generally herein identified by reference E, as shown in FIG. 2.

The device D includes a free-standing tripod 10, a rack 12 at an upper end of the tripod 10, and a drying unit 14. The tripod 10 includes a lower base or stand 16 and a central mast 18 extending above the stand 16. The stand 16 has three angled legs 20 each provided with a floor support 22 that can be anchored to the floor using appropriate fasteners (not shown) extending through openings 24 defined the floor support 22.

The mast 18 has upper and lower sections 26 and 28 joined by a connector 30.

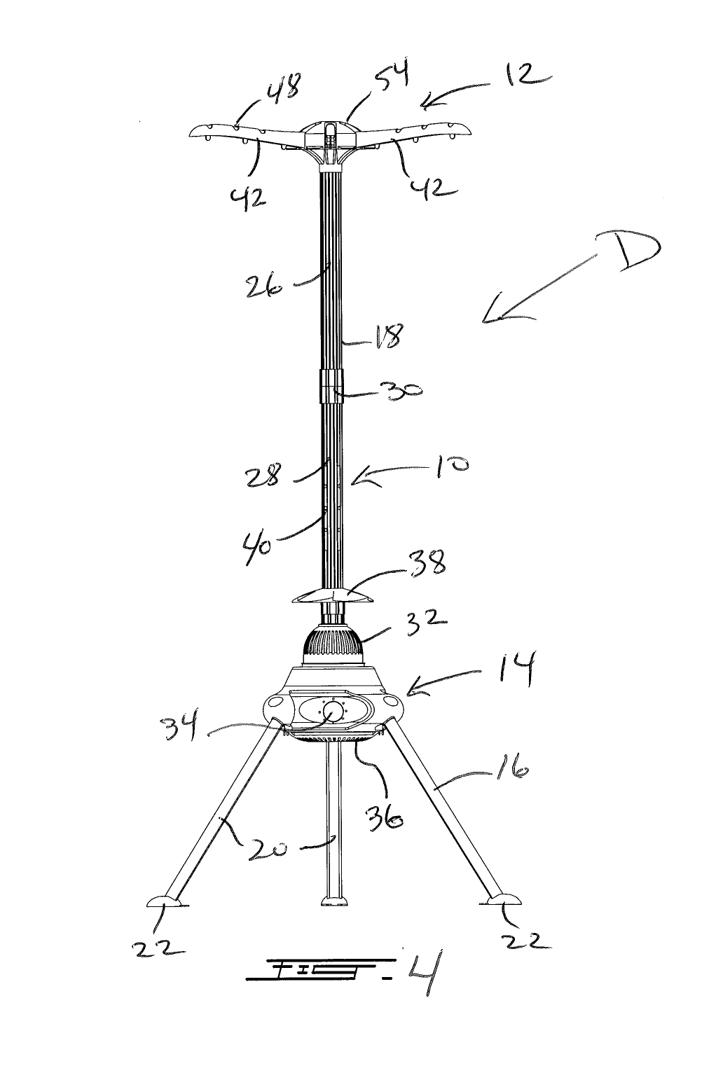

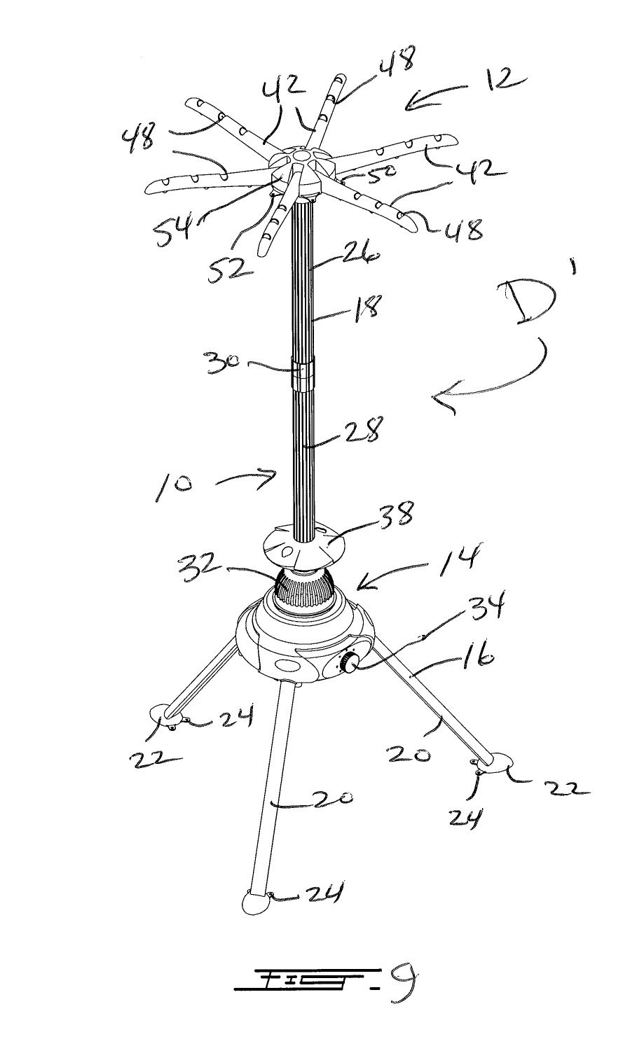

The drying unit 14 includes a blower or fan adapted to force air through an air outlet 32 and upwardly towards the rack 12. The drying unit 14 is provided with a timer 34 so that the blower can operate for a pre-set desired time period. An air entry 36 for the blower is provided at a lower end of the drying unit 14, as seen in FIGS. 4 and 8.

Typically, the drying unit 14 includes a heating system such that the air conveyed by the drying unit 14 to the equipment E is warm. A waterproof plate 38 is provided on a lower end of the mast 18 and above the drying unit 14 for preventing water dripping from the equipment E from accessing the drying unit 14 via the air outlet 32 thereof.

An ultraviolet (UV) light 40 is provided on the mast 18, above the waterproof plate 38 for sanitizing the equipment E. The electrical cable for the blower of the drying unit 14 and for the UV light 40 is not herein shown.

The rack 12 includes a series of radially and horizontally extending arms 42, from which the equipment E can be hung. The arms 42 define upper recesses or notches 48 so that the equipment E can be hung therefrom with more stability. Tabs 50 are provided between the arms 42, with each tab 50 defining a hole 52. S-hooks 56 (FIG. 2) are also provided from hanging the equipment E from the rack 12, which for instance can be engaged in the holes 52 or in the notches 48. The arms 42 are pivotally mounted to a central hub 54 of the rack 12 for storage purposes.

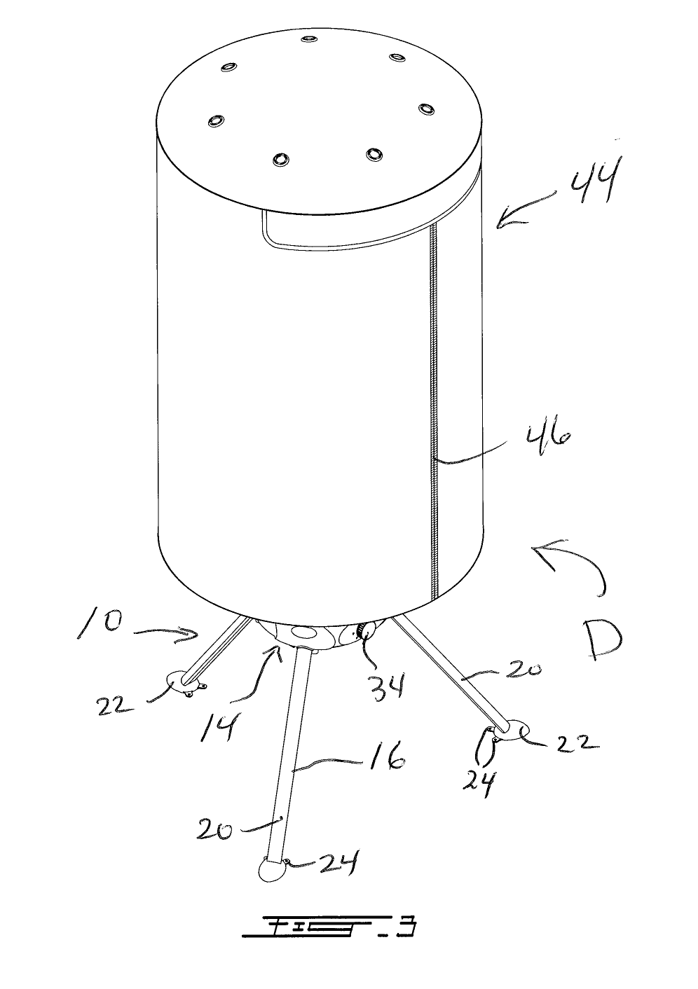

The device D also includes a bag or cover 44 (see FIG. 3) provided with a side zipper 46. The cover 44 is adapted to substantially encapsulate the equipment E hanging from the rack 12 so that the heated air and the UV light or ozonator 40 are mostly contained in within the cover 44 and act efficiently on the equipment E. The cover 44 is open-ended at its bottom end and this bottom end can be attached around the drying unit 14, below the air outlet 32 and above the air entry 36.

The drying unit 14, using a positive temperature co-efficient heating element, and the UV light 40 reduce smell and kill bacteria in the equipment E.

Therefore, sporting equipment and clothing E can be hung from the rack 12 and within the bag 44 so as to be dried by air provided by the forced air motor of the drying unit 14, and can be sanitized by the warm air and the UV light 40.

The device D′ of FIG. 9 is similar to the device D of FIGS. 1 to 8, except that it does not include the UV light 40.

While the above description provides examples of the embodiments, it will be appreciated that some features and/or functions of the described embodiments are susceptible to modification without departing from the spirit and principles of operation of the described embodiments. Accordingly, what has been described above has been intended to be illustrative and non-limiting and it will be understood by persons skilled in the art that other variants and modifications may be made without departing from the scope of the invention as defined in the claims appended hereto.

1. A drying device for items such as sporting equipment and clothing, comprising a free-standing structure, a rack at an upper end of the free-standing structure, a forced air motor at a lower end of the free-standing structure, and an enclosure extending outwardly of the rack and downwardly towards the forced air motor, sporting equipment and/or clothing being adapted to be hung from the rack and inwardly of the enclosure, whereby air provided by the forced air motor flows on the sporting equipment and/or clothing thereby at least partly drying the same.

2. A drying device as defined in claim 1, wherein a heating unit is provided such that the air from the forced air motor is warm.

3. A drying device as defined in claim 1, wherein a UV light is provided within the enclosure.

4. A drying device for items, comprising a support structure, a rack at an upper end of the structure, a blower at a lower end of the structure, and an enclosure extending outwardly of the rack and downwardly towards the blower, the items being adapted to be hung from the rack and inwardly of the enclosure, whereby air provided by the blower acts on the items for at least partly drying the same.

5. A drying device as defined in claim 4, wherein the blower is adapted to produces heated air.

6. A drying device as defined in claim 4, wherein a UV light is provided within the enclosure.
